(19) United States
(12) Patent Application Publication
(10) Pub. No.: US 2023/0031893 A1
(43) Pub. Date: Feb. 2, 2023

(54) SYSTEM AND A METHOD FOR CONTROLLING OXYGEN SUPPLY EOUIPMENTS

(71) Applicant: L&T TECHNOLOGY SERVICES LIMITED, Chennai (IN)

(72) Inventors: Jagdish Prasad Sahu, Mysore (IN); Ravi Kumar, Tumkur (IN); Samyuktha Bangalore Srinivas, Bangalore (IN); Balakrishnarao Manali Srinivas, Mysore (IN); Ashish Kumar, Godda (IN); Rishabh Ranjan, Patna (IN); Keyur Bhalodiya, Rajkot (IN)

(21) Appl. No.: 17/694,957

(22) Filed: Mar. 15, 2022

(30) Foreign Application Priority Data
Jul. 30, 2021 (IN) 202141034304

(51) Int. Cl. A61M 16/20; A61M 16/10; A61M 16/00

(52) U.S. Cl. CPC A61M 16/202; A61M 16/1005; A61M 16/0051; A61M 16/0003; A61M 2016/0027; A61M 2016/0018; A61M 2016/003

(57) ABSTRACT

Disclosed herein is a system and method for controlling oxygen supply equipments. The system is equipped with pressure sensors coupled to the oxygen supply equipments. Depending on the oxygen supply selected by a user for the patient, the pressure sensor monitors the current pressure of the preset oxygen supply during an inhalation phase corresponding to the patient. If the current pressure is less than a minima value or if a pressure drop rate is higher than or equal to a threshold rate or both, the system generates an alarm notifying the user to check the preset oxygen supply and in the meanwhile switches the preset oxygen supply from one oxygen supply equipment to another oxygen supply equipment or vice-versa.

### TECHNICAL FIELD

The present disclosure relates generally to ventilator systems and more particularly to providing a system and method for controlling oxygen supply equipments.

### BACKGROUND

The following description includes information that may be useful in understanding the present disclosure. It is not an admission that any of the information provided herein is prior art or relevant to the presently claimed disclosure, or that any publication specifically or implicitly referenced is prior art.

Ventilator systems are very commonly used to deliver a fluid, such as oxygen, air, or other breathing gas or gas mixture, to an airway of patient to augment, supplement, or substitute the patient's own ventilatory effort and/or to treat the patient with a pressure support therapy. In such systems, it is highly important that a continuous flow of the fluid (hereinafter referred to as “oxygen”) at a required pressure is maintained in order to ensure that the patient in need does not suffer due to either disrupt in the flow of oxygen or fluctuating pressure.

### SUMMARY

The following presents a simplified summary to provide a basic understanding of some aspects of the disclosed material handling system. This summary is not an extensive overview and is intended to neither identify key or critical elements nor delineate the scope of such elements. Its purpose is to present some concepts of the described features in a simplified form as a prelude to the more detailed description that is presented later.

Various example embodiments described herein relate to a system for controlling oxygen supply. The system includes a processor and a memory, wherein the processor is communicably coupled to the memory, a first oxygen supply equipment and a second oxygen supply equipment. The processor configured to: determine a pressure of oxygen supply by using one or more sensors coupled to at least one of the first oxygen supply equipment or the second oxygen supply equipment during an inhalation phase and compare the pressure of the oxygen supply with a pressure threshold associated with one of the first oxygen supply equipment or the second oxygen supply equipment. In response to the comparison, actuate at least a valve coupled to the first oxygen supply equipment or the second oxygen supply equipment to switch the oxygen supply from the first oxygen supply equipment to the second oxygen supply equipment or vice-versa when the oxygen supply falls below the pressure threshold.

Various example embodiments described herein relate to a system for controlling oxygen supply, wherein the pressure threshold is indicative of a low-pressure minima associated with first oxygen supply equipment and a high-pressure minimum associated with the second oxygen supply equipment.

Various example embodiments described herein relate to a system for controlling oxygen supply, wherein pressure range of the oxygen supply from the first oxygen supply equipment is about approximately 5 to 7 Psi.

Various example embodiments described herein relate to a system for controlling oxygen supply, wherein pressure range of the oxygen supply from the first oxygen supply equipment is about approximately 5-30 Psi.

Various example embodiments described herein relate to a system for controlling oxygen supply, wherein the processor is further configured to: determine a pressure drop rate and compare the pressure drop rate with a pressure drop rate threshold; wherein when the pressure drop rate exceeds or is equal to the pressure drop rate threshold and switch the oxygen supply from the first oxygen supply equipment to the second oxygen supply equipment or vice-versa.

Various example embodiments described herein relate to a system for controlling oxygen supply, wherein the processor is further communicatively coupled to an alarm device and configured to generate an alarm when at least one of: the pressure falls below the pressure threshold; or the pressure drop rate exceeds the pressure drop rate threshold.

Various example embodiments described herein relate to a system for controlling oxygen supply, wherein the processor is further configured to: regulate the pressure of the oxygen supply to first oxygen supply equipment and the second oxygen supply equipment via a pressure regulator using feedback from the one or more sensors.

Various example embodiments described herein relate to a system for controlling oxygen supply, wherein the memory further stores patient related data and oxygen supply equipment related data, wherein: the patient related data comprises at least one of patient name, patient age, patient address, patient ailment history and patient treatment history; and the oxygen supply equipment related data comprises at least one of nominal oxygen content, nominal outlet pressure, flow rate and net weight.

Various example embodiments described herein relate to a system for controlling oxygen supply, wherein the processor is further configured to: select one of the first oxygen supply equipment or the second oxygen supply equipment to be used on the patient based on the patient related data.

Various example embodiments described herein relate to a system for controlling oxygen supply, wherein the processor is further configured to: preset the pressure threshold of the first oxygen supply equipment and the second oxygen supply equipment based on the oxygen supply equipment related data and the patient related data.

Various example embodiments described herein relate to a method for controlling oxygen supply. The method includes detecting current pressure of a preset oxygen supply during each inhalation phase of a patient, wherein the preset oxygen supply is provided by either a high-flow oxygen supply equipment or a low-flow oxygen supply equipment. The high-flow oxygen supply equipment is capable of supplying oxygen in a high-pressure range and the low-flow oxygen supply equipment is capable of supplying oxygen in a low-pressure range. The method further includes comparing the current pressure of the preset oxygen supply with either a low-pressure minimum associated with the low-pressure range or a high-pressure minima associated with the high-pressure range. Based on comparison, switching the preset oxygen supply from the low-flow oxygen supply equipment to the high-flow oxygen supply equipment when the current pressure falls below the low-pressure minima or switching the preset oxygen supply from the high-flow oxygen supply equipment to the low-flow oxygen supply equipment when at least one of: the current pressure falls below the high-pressure minima and a pressure drop rate of the current pressure exceeds or is equal to a pressure drop rate threshold.

Various example embodiments described herein relate to a method for controlling oxygen supply, wherein both the current pressure falling below the high-pressure minima and the pressure drop rate of the current pressure exceeding the pressure drop rate threshold are indicative of emptying of the high-flow oxygen supply equipment.

Various example embodiments described herein relate to a method for controlling oxygen supply, wherein detecting the current pressure further comprises detecting the current pressure of the preset oxygen supply by a low-pressure sensor when the preset oxygen supply is provided by the low-flow oxygen supply equipment and detecting the current pressure of the preset oxygen supply by a high-pressure sensor when the preset oxygen supply is provided by the high-flow oxygen supply equipment.

Various example embodiments described herein relate to a method for controlling oxygen supply, further comprising generating an alarm when at least one of: the current pressure falls below the low-pressure minima; the current pressure falls below the high-pressure minima; and the pressure drop rate exceeds the pressure drop rate threshold.

Various example embodiments described herein relate to a method for controlling oxygen supply, wherein switching between the high-flow oxygen supply equipment and the low-flow oxygen supply equipment further comprises actuating a valve connected with both the high-flow oxygen supply equipment and the low-flow oxygen supply equipment.

Various example embodiments described herein relate to a method for controlling oxygen supply, further comprising selecting the preset oxygen supply based on patient related data, and wherein the patient related data comprises at least one of patient name, patient age, patient address, patient ailment history and patient treatment history.

Various example embodiments described herein relate to a method for controlling oxygen supply, further comprising selecting one of the high-flow oxygen supply equipment and the low-flow oxygen supply equipment to be used on the patient based on the patient related data.

Various example embodiments described herein relate to a method for controlling oxygen supply, further comprising selecting the preset oxygen supply based on oxygen supply equipment, and wherein the oxygen supply equipment related data comprises at least one of nominal oxygen content, nominal outlet pressure, flow rate and net weight.

Various example embodiments described herein relate to a method for controlling oxygen supply, further comprising regulating the pressure of the oxygen supply to first oxygen supply equipment and the second oxygen supply equipment.

Various example embodiments described herein relate to a method for controlling oxygen supply, further comprising simultaneously generating the alarm and actuating the valve when at least one of: the current pressure falls below the low-pressure minima; the current pressure falls below the high-pressure minima; and the pressure drop rate exceeds or equals the pressure drop rate threshold.

The above summary is provided merely for purposes of summarizing some example embodiments to provide a basic understanding of some aspects of the disclosure.

Accordingly, it will be appreciated that the above-described embodiments are merely examples and should not be construed to narrow the scope or spirit of the disclosure in any way. It will be appreciated that the scope of the disclosure encompasses many potential embodiments in addition to those here summarized, some of which will be further described below.

### BRIEF DESCRIPTION OF DRAWINGS

The embodiments of the disclosure itself, as well as a preferred mode of use, further objectives and advantages thereof, will best be understood by reference to the following detailed description of an illustrative embodiment when read in conjunction with the accompanying drawings. One or more embodiments are now described, by way of example only, with reference to the accompanying drawings in which:

FIG. 1A depicts an exemplary environment of a system for controlling oxygen supply equipments, in accordance with an embodiment of the present disclosure.

FIG. 1B depicts an exemplary environment of a system for controlling at least two oxygen supply equipment, in accordance with an embodiment of the present disclosure.

FIG. 2 depicts a block diagram of a system for controlling oxygen supply equipment, in accordance with an embodiment of the present disclosure.

FIG. 3 depicts a flowchart of a method for controlling oxygen supply equipment, in accordance with an embodiment of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

Various embodiments of the present invention now will be described more fully hereinafter with reference to the accompanying drawings, in which some, but not all embodiments of the invention are shown. Indeed, the invention may be embodied in many different forms and should not be construed as limited to the embodiments set forth herein.

Rather, these embodiments are provided so that this disclosure will satisfy applicable legal requirements. The term “or” is used herein in both the alternative and conjunctive sense, unless otherwise indicated. The terms “illustrative,” “example,” and “exemplary” are used to be examples with no indication of quality level. Like numbers refer to like elements throughout.

The phrases “in an embodiment,” “in one embodiment,” “according to one embodiment,” and the like generally mean that the particular feature, structure, or characteristic following the phrase may be included in at least one embodiment of the present disclosure and may be included in more than one embodiment of the present disclosure (importantly, such phrases do not necessarily refer to the same embodiment).

The word “exemplary” is used herein to mean “serving as an example, instance, or illustration.” Any implementation described herein as “exemplary” is not necessarily to be construed as preferred or advantageous over other implementations.

If the specification states a component or feature “can,” “may,” “could,” “should,” “would,” “preferably,” “possibly,” “typically,” “optionally,” “for example,” “often,” or “might” (or other such language) be included or have a characteristic, that particular component or feature is not required to be included or to have the characteristic.

Such component or feature may be optionally included in some embodiments, or it may be excluded.

Many types of ventilator systems available in the market based on the type of oxygen supplies compatible with the ventilator systems. For example, some ventilator systems only support oxygen cylinders (hereinafter referred to as “high-flow oxygen supply equipment”) while others only support oxygen concentrators (hereinafter referred to as “low-flow oxygen supply equipment”). Also available are those ventilators that can support both high-flow and low-flow oxygen supply equipments. With any of the types of ventilator systems, the major objective is to ensure a continuous supply of oxygen to the patient at a required pressure level. However, there can be many issues that can hinder a continuous oxygen supply to the patient, such as, a fault in the oxygen supply line or leakage that may result in reduced pressure or emptying of the oxygen supply equipments. Therefore, while administering oxygen supply to a patient, a medical professional must cautiously and timely monitor the patient. This might require tedious effort and might often result in situations that may be detrimental to the health of the patients and in worst cases, their survival. Moreover, if multiple oxygen supply equipments are being used, it becomes more challenging in managing and controlling the oxygen supply one vis-à-vis the other.

Conventional systems employing multiple oxygen supply equipments for the patients encounter certain shortcomings. For example, the supply of oxygen may be monitored regularly by a medical professional to ensure that the patient is receiving continuous supply of oxygen. In such a scenario, a negligence at the end of the medical professional can prove detrimental to the well-being of the patient. Further, even if the medical professional is diligent in monitoring the patient, situations like rapid falling of oxygen pressure due to emptying of the oxygen supply equipment is out of his/her control. Furthermore, conventional systems lack provisions of detecting a fault or a leakage or emptying of the oxygen supply equipment.

Through applied effort, ingenuity, and innovation, many of the above identified problems have been solved by developing solutions that are included in embodiments of the present disclosure, many examples of which are described in detail herein. The present disclosure relates to a system and method for controlling oxygen supply equipments. A pressure of oxygen supply is determined by using one or more sensors coupled to at least one of the first oxygen supply equipment or the second oxygen supply equipment during an inhalation phase. The pressure of the oxygen supply is compared with a pressure threshold associated with one of the first oxygen supply equipment or the second oxygen supply equipment. In response to the comparison, at least a valve coupled to the first oxygen supply equipment or the second oxygen supply equipment is actuated to switch the oxygen supply from the first oxygen supply equipment to the second oxygen supply equipment or vice-versa when the oxygen supply falls below the pressure threshold. The automatic switching between the oxygen supply equipments may ensure that a continuous supply of oxygen is provided at all times to the patient. Further, an alarm is generated when the pressure falls below the pressure threshold which provides a buffer time to the medical professional to check the reason for the alarm and take necessary actions while ensuring that the patient receives continuous oxygen supply even while the preset oxygen supply is being checked by the medical professional.

According to an embodiment, the pressure threshold is indicative of a low-pressure minima associated with first oxygen supply equipment and a high-pressure minimum associated with the second oxygen supply equipment.

According to an embodiment, a pressure drop rate from the at least one oxygen supply equipment is determined and compared with a pressure drop rate threshold; wherein when the pressure drop rate exceeds or is equal to the pressure drop rate threshold, the oxygen supply is switched from the first oxygen supply equipment to the second oxygen supply equipment or vice-versa.

According to an embodiment, the alarm is generated, and the valve is actuated when at least one of: the current pressure falls below the low-pressure minima; the current pressure falls below the high-pressure minima; or the pressure drop rate exceeds or equals the pressure drop rate threshold

In the following detailed description of exemplary embodiments of the disclosure, specific representative embodiments in which the disclosure may be practiced are described in sufficient detail to enable those skilled in the art to practice the disclosed embodiments.

For example, specific details such as specific method orders, structures, elements, and connections have been presented herein. However, it is to be understood that the specific details presented need not be utilized to practice embodiments of the present disclosure. It is also to be understood that other embodiments may be utilized and that logical, architectural, programmatic, mechanical electrical and other changes may be made without departing from the general scope of the disclosure. The following detailed description is, therefore, not to be taken in a limiting sense, and the scope of the present disclosure is defined by the appended claims and equivalents thereof.

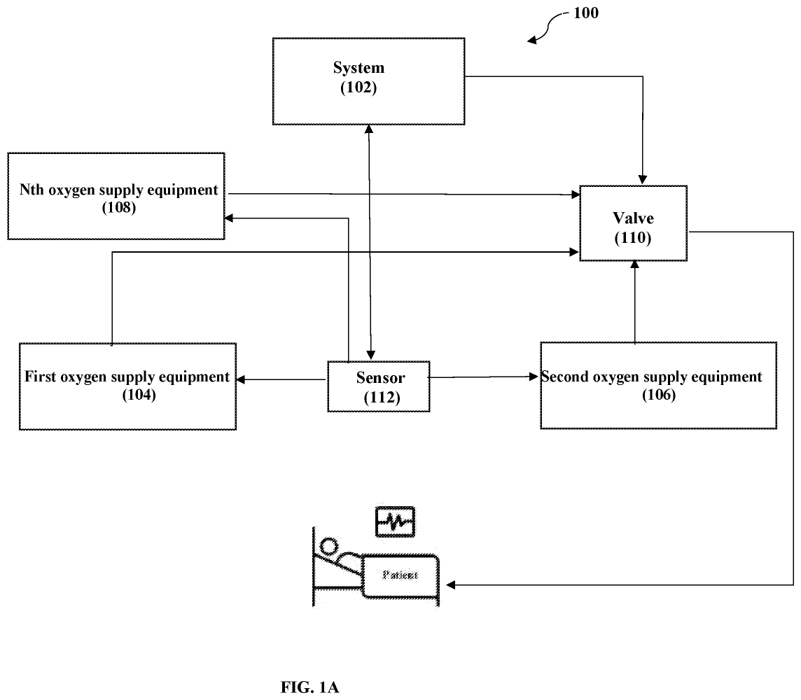

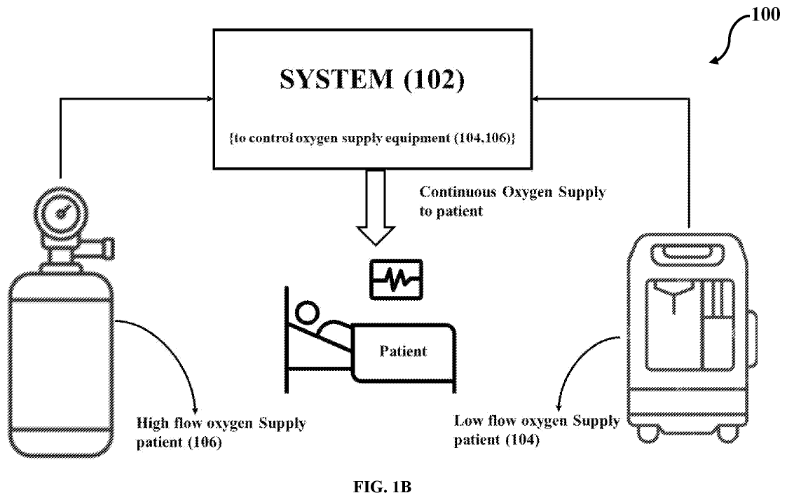

FIG. 1A depicts an exemplary environment of a system for controlling oxygen supply equipment, in accordance with an embodiment of the present disclosure. According to an embodiment, the environment includes a system coupled with one or more oxygen supply equipments. The system may include a processor and a memory, wherein the processor is communicably coupled to the memory and the one or more oxygen supply equipments. According to an embodiment, the processor may determine a pressure of oxygen supply using at least one and control the one or more oxygen supply equipments using at least one valve based on the determined pressure. In some examples, the processor may be implemented as one or more microprocessors, microcomputers, microcontrollers, digital signal processors, central processing units, state machines, logic circuitries, and/or any devices that manipulate signals based on operational instructions. Among other capabilities, the processor may be configured to fetch and execute computer-readable instructions stored in the memory.

According to an embodiment, the memory stores a pressure threshold and a pressure drop rate threshold associated with each of the oxygen supply equipment, in the environment. According to an embodiment, the pressure threshold and the pressure drop rate threshold may be pre-set by a user or the processor based on the oxygen supply equipment data and patient's requirement, which will be discussed later in the description. In some examples, the pre-set threshold values may exist in the form a look-up table in which each oxygen supply equipment, is associated with at least one threshold value. In some examples, the threshold value may also be specified in the memory as a threshold value range. By way of example but not limitation, the memory may include random access memory (RAM), read-only memory (ROM), electrically erasable programmable read-only memory (EEPROM), FLASH memory, disk storage, magnetic storage devices, or the like. Disk storage, as used herein, includes compact disc (CD), laser disc, optical disc, digital versatile disc (DVD), floppy disk, and Blu-ray Disc™, or other storage devices that store data magnetically or optically with lasers. Combinations of the above types of media are also included within the scope of the terms non-transitory computer-readable and processor-readable media. Additionally, any combination of instructions stored on the one or more non-transitory processor-readable or computer-readable media may be referred to herein as a computer program product.

In some examples, the at least one sensor may be a gas pressure sensor which can be designed or can be configured to measure gas pressure in multiple ways. By way of example but not limitation, the gas pressure sensor may be gauge pressure sensor, sealed gas pressure sensor, absolute gas pressure, differential pressure sensor and the like known to a person skilled in the art.

In some examples, the at least one valve may be 2/3-way ball valve, 2/3-way solenoid valve, 2/3-way switching/diverting/changeover valve. The valve may be actuated either electrically or pneumatically or hydraulically as known to a person skilled in the art.

According to an embodiment, as shown in FIG. 1A, the one or more oxygen supply equipments may be a first oxygen supply equipment, a second oxygen supply equipment and Nth oxygen supply equipment, wherein N represents the number of oxygen supply equipment that can be coupled to the system. Each of the oxygen supply equipment, may be capable of supplying oxygen at a required pressure to the patient. In some examples, the first oxygen supply equipment may be considered as a primary oxygen supply equipment to supply oxygen to the patient while the other oxygen supply equipment (i.e., the second oxygen supply equipment up to Nth oxygen supply equipment may be considered as a secondary oxygen supply equipment which may serve as emergency back up in the event of any interruptions in the oxygen supply by the primary oxygen supply equipment.

According to an embodiment, the processor may determine the pressure of the oxygen supplied by the one or more oxygen supply equipments, (i.e., the primary oxygen supply equipment and the secondary oxygen supply equipment) during each inhalation phase of the patient by using one or more sensors. In some examples, each oxygen supply equipment, may be communicably coupled to at least one sensor such that the pressure of oxygen supply during each inhalation phase of the patient may be determined and communicated to the system for further processing. According to an embodiment, the pressure may be continuously broadcasted via a communication channel to the system at regular predefined intervals, however, conceivable are other embodiments in which the system may query via the communication channel, the at least one sensor of the oxygen supply equipment, currently supplying the oxygen to the patient. In this regard, the system may selectively monitor the pressure of only the oxygen supply, which are actively supplying the oxygen to the patient. For example, the system may monitor only the primary oxygen supply equipment which is actively supplying the oxygen to the patient rather than the secondary oxygen supply equipment.

According to an embodiment, the processor may compare the pressure sensed using the at least one sensor with the pressure threshold during each inhalation phase of the patient. As previously discussed, the at least one sensor may be capable of broadcasting current pressure readings of either the first oxygen supply equipment or the second oxygen supply equipment or both. The current pressure readings may be compared with the pre-set pressure threshold. Such a comparison may be required to check whether the supply of oxygen to patient is at the required pressure levels.

According to an embodiment, as a result of the comparison, the processor actuates at least one valve coupled to the first oxygen supply equipment or the second oxygen supply equipment to switch the oxygen supply from the first oxygen supply equipment to the second oxygen supply equipment or vice-versa when the oxygen supply falls below the pressure threshold. For example, the processor may transmit a control signal to the actuator of the at least one valve to switch the flow of oxygen supply from one oxygen supply equipment to another oxygen supply equipment. In some examples, the at least one valve may be a 3-way switching valve assembly can also be controlled by pneumatic or electric actuators with two input ports and one output port, wherein a first input port may be connected to the first oxygen supply equipment and second input port may be connected to the second oxygen supply equipment. In operation, when the current pressure reading of the oxygen flow of the first input port is less than the preset pressure threshold, the processor actuates the valve such that the first input port is closed and the second input port is open, thereby switching the oxygen flow from the first oxygen supply equipment to the second oxygen supply equipment. In this regard, automatic switching is performed to continuously supply the patient with oxygen even when one of the oxygen supply equipment is emptied or at fault.

According to an embodiment, each of the oxygen supply equipment may include at least one valve to control the oxygen flow. For example, the at least one valve may be a 2-way solenoid valve which uses a plunger to open or close the valve. For example, the first oxygen supply equipment may include a first solenoid valve and the second oxygen supply equipment may include a second solenoid valve. In operation, when the current pressure reading of the oxygen flow of the first input port is less than the preset pressure threshold, the processor may shut down the first solenoid valve and switch on the second solenoid valve. In other words, a power to the first solenoid valve is shut down causing the plunger to be pulled down, effectively sealing the valve and preventing the flow of oxygen. At the same time, the power to the second solenoid valve is switched on causing the plunger to rise, effectively unsealing the valve and allowing the flow of oxygen. In this regard, automatic switching is performed to continuously supply the patient with oxygen even when one of the oxygen supply equipment is emptied or at fault.

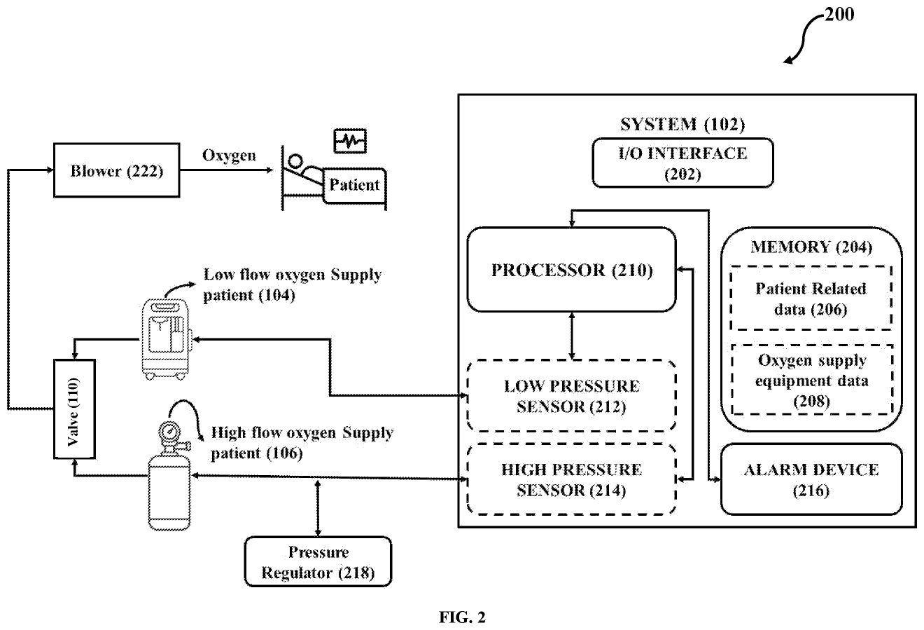

According to an embodiment, the pressure threshold may be indicative of a low-pressure minima associated with first oxygen supply equipment and a high-pressure minima associated with the second oxygen supply equipment. Such pressure minima values and ranges used as pressure thresholds will be explained in detailed in conjunction with embodiments discussed with FIG. 1B and FIG. 2

According to an embodiment, the automatic switching is performed based on a comparison between a pressure drop rate associated with the oxygen supply equipment and a pressure drop rate threshold. The processor may determine a pressure drop rate; compare the pressure drop rate with the pressure drop rate threshold; and switch the oxygen supply from the first oxygen supply equipment to the second oxygen supply equipment or vice-versa when the pressure drop rate exceeds or is equal to the pressure drop rate threshold. These and other embodiments are explained in detailed in conjunction with FIG. 1B and FIG. 2.

According to an embodiment, an alarm may be generated while the processor actuates the valve in order to switch the oxygen supply from one oxygen supply equipment to another oxygen supply equipment. These and other embodiments are explained in detailed in conjunction with FIG. 1B and FIG. 2.

It is well understood by a skilled person that the embodiments are discussed with reference to two oxygen supply equipments for ease of explanation. Therefore, it is to be understood that the solution proposed in present disclosure are not to be limited to two oxygen supply equipments rather is suitable for use with a plurality of oxygen supply equipments.

FIG. 1B depicts an exemplary environment of a system for controlling at least two oxygen supply equipment, in accordance with an embodiment of the present disclosure. It must be noted by a skilled person that the exemplary environment may also be implemented in various environments, other than as shown in FIG. 1B. The exemplary environment is explained in conjunction with FIG. 2 that shows a block diagram of a system, in accordance with an embodiment of the present disclosure.

In one implementation, the system may comprise an I/O interface, the memory, the processor, a low-pressure sensor, a high-pressure sensor and an alarm device. The memory may be communicatively coupled to the processor. The processor may be communicatively coupled to the low-pressure sensor, the high-pressure sensor, and the alarm device. According to an embodiment, the low-pressure sensor, the high-pressure sensor, and the alarm device may be external to the system. Further, the memory may store patient related data and oxygen supply equipment data. The significance and use of each of the stored quantities is explained in the subsequent paragraphs.

The I/O interface may include a variety of software and hardware interfaces, for example, a web interface, a graphical user interface, and the like. The I/O interface may enable the system to communicate with other computing devices, such as web servers and external data servers (not shown). The I/O interface may facilitate multiple communications within a wide variety of networks and protocol types, including wired networks, for example, LAN, cable, etc., and wireless networks, such as WLAN, cellular, or satellite. The I/O interface may include one or more ports for connecting many devices to one another or to another server.

According to the embodiments discussed in conjunction with FIG. 1B and FIG. 2, the first oxygen supply equipment may be a low-flow oxygen supply equipment and the second oxygen supply equipment may be a high-flow oxygen supply equipment. It is well understood by a skilled person those other alternative arrangements in which both first oxygen supply equipment and the second oxygen supply equipment are high-flow oxygen supply equipments or low-flow oxygen supply equipments falls within the scope of the present disclosure.

In one implementation, the low-pressure sensor is communicatively coupled to the low-flow oxygen supply equipment and the high-pressure sensor is communicatively coupled to the high-flow oxygen supply equipment. The low-flow oxygen supply equipment is capable of supplying oxygen in a low-pressure range while the high-flow oxygen supply equipment is capable of supplying oxygen in the high-pressure range. In accordance with the exemplary embodiment, the low-flow oxygen supply equipment is capable of supplying oxygen in a low-pressure range of 5-7 Psi., while the high-flow oxygen supply equipment in capable of supplying oxygen in a high-pressure range of 5-30 Psi. Such information about the oxygen supply equipment along with other data such as nominal oxygen content, nominal outlet pressure, flow rate and net weight is stored as oxygen supply equipment data in the memory.

In one implementation, the low-flow oxygen supply equipment and the high-flow oxygen supply equipment are connected to a valve which is further connected to a blower configured to deliver oxygen to the patient. In one embodiment, the valve is a 3/2-type solenoid valve and is configured to actuate in order to allow flow of oxygen from either the low-flow oxygen supply equipment or the high-flow oxygen supply equipment at a given point of time. However, it may be noted by a skilled person that any other suitable type of valve may also be used for the purpose of actuation. Further, in one embodiment, a pressure regulator is provided between the high-pressure sensor and the high-flow oxygen supply equipment in order to regulate the pressure of oxygen supply being released by the high-flow oxygen supply equipment. In one implementation, the wherein the processor may regulate the pressure of the oxygen supply to the low-flow oxygen supply equipment and the high-flow oxygen supply equipment via the pressure regulator using feedback from the low-pressure sensor and the high-pressure sensor.

Now, referring to FIG. 1B, the environment shows that the system controls the low-flow oxygen supply equipment and the high-flow oxygen supply equipment in order to ensure a continuous supply of oxygen to the patient. The working of the system begins when a medical professional (hereinafter referred to as “user”) selects an oxygen supply to be delivered to the patient based on the patient related data stored in the memory. In one embodiment, the patient related data comprises patient's name, age, address, ailment data and treatment data. In the starting, the oxygen supply to be delivered to the patient may be provided by either the low-flow oxygen supply equipment or the high-flow oxygen supply equipment depending on the user's selection. In one embodiment, the processor is configured to select one of the low-flow oxygen supply equipment and the high-flow oxygen supply equipment to be used on the patient based on the patient related data.

In one embodiment, it is considered that the user selects low-flow oxygen supply equipment based on the patient related data. In this case the preset oxygen supply is provided by the low-flow oxygen supply equipment. The user also sets a low-pressure minimum based on the oxygen supply equipment data and patient's requirement. However, it may be noted that the low-pressure minima is always set at a value greater than what is actually required by the patient to be at safer side. For instance, if the patient's requirement is 3 Psi oxygen pressure and the low-flow oxygen supply equipment is capable of providing oxygen within a pressure range of 5-7 Psi, the low-pressure minima may be set as 5 Psi. In one embodiment, the processor is configured to preset the high-pressure minima and low-pressure minima based on the oxygen supply equipment related data and the patient related data.

Once the user selects low-flow oxygen supply equipment, the processor that is communicatively coupled to the valve, actuates the valve so as to allow oxygen supply only from the low-flow oxygen supply equipment. As the preset oxygen supply flows to the patient, the low-pressure sensor detects the current pressure of the preset oxygen supply during each inhalation phase corresponding to the patient. In other words, the current pressure is detected only when the patient inhales oxygen being provided by the low-flow oxygen supply equipment. Upon detecting the current pressure, the processor compares the current pressure with the low-pressure minima. If the current pressure is detected to be below the low-pressure minima, the processor sends a signal to the alarm device to generate an alarm and in the meanwhile simultaneously actuates the valve in order to switch the preset oxygen supply to the high-flow oxygen supply equipment. The pressure from the high-flow oxygen supply equipment may be regulated by the pressure regulator in order to meet the required pressure. In this way, while the user checks the reason for alarm, in terms of either a fault in the low-flow oxygen supply equipment or emptying of the low-flow oxygen supply equipment, the patient is continuously supplied with oxygen. The switching, therefore, provides a buffer time to the user to rectify the situation, which is to replace the low-flow oxygen supply equipment etc., without being worried about the well-being of the patient.

In another embodiment, it is considered that the user selects high-flow oxygen supply equipment based on the patient related data. In this case, the preset oxygen supply is provided by the high-flow oxygen supply equipment. The user also sets a high-pressure minima based on the oxygen supply equipment data and patient's requirement. However, it may be noted that the high-pressure minima is always set at a value greater than what is actually required by the patient. For instance, if the patient's requirement is 15 Psi oxygen pressure and the high-flow oxygen supply equipment is capable of providing oxygen within a pressure range of 5-30 Psi, the high-pressure minima may be set as 20 Psi. Further, the user also sets a pressure drop rate minima. For instance, in one embodiment, the pressure drop rate threshold may be set as 0.5 Psi/min. In one embodiment, the processor is configured to preset the high-pressure minima and pressure drop rate minima based on the oxygen supply equipment () related data and the patient related data ().

Once the user selects high-flow oxygen supply equipment, the processor that is communicatively coupled to the valve, actuates the valve so as to allow oxygen supply only from the high-flow oxygen supply equipment. As the preset oxygen supply flows to the patient, the high-pressure sensor detects the current pressure of the preset oxygen supply during each inhalation phase corresponding to the patient. In other words, the current pressure is detected only when the patient inhales oxygen being provided by the high-flow oxygen supply equipment. The high-pressure sensor also detects a pressure drop rate of the preset oxygen supply. Upon detecting the current pressure, the processor compares the current pressure with the high-pressure minima and simultaneously compares the pressure drop rate with the pressure drop rate threshold. If the current pressure goes below the high-pressure minima or the pressure drop rate becomes higher than the pressure drop rate threshold or a combination of both, the processor sends a signal to the alarm device to generate an alarm and in the meanwhile actuates the valve in order to switch the preset oxygen supply to the low-flow oxygen supply equipment. In this way, while the user checks the reason for alarm, in terms of either a leakage/fault in the high-flow oxygen supply equipment or emptying of the high-flow oxygen supply equipment, the patient is continuously supplied with oxygen. This switching mechanism, therefore, provides a buffer time to the user to rectify the situation, which is to replace the high-flow oxygen supply equipment etc., without being worried about the well-being of the patient.

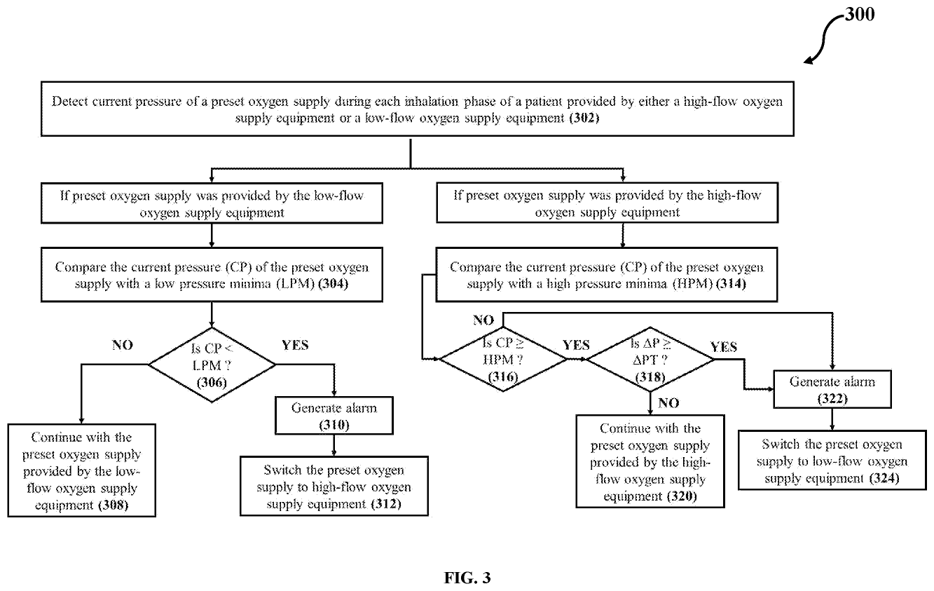

FIG. 3 depicts a method for controlling oxygen supply equipment, in accordance with an embodiment of the present disclosure. As illustrated in FIG. 3, the method includes one or more blocks illustrating a method for controlling oxygen supply equipment. The method may be described in the general context of computer executable instructions. Generally, computer executable instructions may include routines, programs, objects, components, data structures, procedures, modules, and functions, which perform specific functions or implement specific abstract data types.

The order in which the method is described is not intended to be construed as a limitation, and any number of the described method blocks may be combined in any order to implement the method. Additionally, individual blocks may be deleted from the methods without departing from the spirit and scope of the subject matter described.

At block, the method may include detecting current pressure of a preset oxygen supply during each inhalation phase of a patient provided by either a high-flow oxygen supply equipment or a low-flow oxygen supply equipment. If the preset oxygen supply is provided by the low-flow oxygen supply equipment, the method proceeds to block and if the preset oxygen supply is provided by the high-flow oxygen supply equipment, the method proceeds to block. According to embodiment, the high-flow oxygen supply equipment () and the low-flow oxygen supply equipment () to be used on the patient and the preset oxygen supply may be selected using either the patient related data or the oxygen supply equipment () related data or both.

At block, the method may include comparing the current pressure (CP) of the preset oxygen supply with a low-pressure minima (LPM). At block, the method may include detecting if the current pressure (CP) is less than the low-pressure minima (LPM). If the result of the detection is NO, the method proceeds to block and if the result of the detection is YES, the method proceeds to block. At block, the method may include continuing and regulating the preset oxygen supply provided by the low-flow oxygen supply equipment if the current pressure (CP) is not less than the low-pressure minima (LPM). At block, the method may include generating an alarm to check the preset oxygen supply provided by the low-flow oxygen supply equipment if the current pressure (CP) is less than the low-pressure minima (LPM). At block, the method may include switching the preset oxygen supply from the low-flow oxygen supply equipment to the high-flow oxygen supply equipment. According to an embodiment, block and, may be executed simultaneously in response to the comparison at block.

At block, the method may include comparing the current pressure (CP) of the preset oxygen supply with a high-pressure minima (HPM). At block, the method may include detecting if the current pressure (CP) is at least equal to the high-pressure minima (HPM). If the result of the detection is YES, the method proceeds to block and if the result of the detection is NO, the method proceeds to block. At block, the method may include detecting if the pressure drop rate (AP) of the preset oxygen supply exceeds or is equal to a pressure drop rate threshold (APT). If the result of the detection is NO, the method proceeds to block and if the result of the detection is YES, the method proceeds to block. At block, the method may include continuing and regulating the preset oxygen supply provided by the high-flow oxygen supply equipment if the pressure drop rate (AP) of the preset oxygen supply does not exceed the pressure drop rate threshold (APT). At block, the method may include generating an alarm to check the preset oxygen supply provided by the high-flow oxygen supply equipment if the current pressure (CP) is less than the high-pressure minima (LPM) or if the pressure drop rate (AP) of the preset oxygen supply exceeds the pressure drop rate threshold (APT) or both. At block, the method may include switching the preset oxygen supply from the high-flow oxygen supply equipment to the low-flow oxygen supply equipment. According to an embodiment, block and, may be executed simultaneously in response to the comparison at block.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

In some example embodiments, certain ones of the operations herein may be modified or further amplified as described below. Moreover, in some embodiments additional optional operations may also be included. It should be appreciated that each of the modifications, optional additions or amplifications described herein may be included with the operations herein either alone or in combination with any others among the features described herein.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The hardware used to implement the various illustrative logics, logical blocks, modules, and circuits described in connection with the aspects disclosed herein may include a general purpose processor, a digital signal processor (DSP), a special-purpose processor such as an application specific integrated circuit (ASIC) or a field programmable gate array (FPGA), a programmable logic device, discrete gate or transistor logic, discrete hardware components, or any combination thereof designed to perform the functions described herein. A general-purpose processor may be a microprocessor, but, in the alternative, the processor may be any processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, or in addition, some steps or methods may be performed by circuitry that is specific to a given function.

In one or more example embodiments, the functions described herein may be implemented by special-purpose hardware or a combination of hardware programmed by firmware or other software. In implementations relying on firmware or other software, the functions may be performed as a result of execution of one or more instructions stored on one or more non-transitory computer-readable media and/or one or more non-transitory processor-readable media. These instructions may be embodied by one or more processor-executable software modules that reside on the one or more non-transitory computer-readable or processor-readable storage media. Non-transitory computer-readable or processor-readable storage media may in this regard comprise any storage media that may be accessed by a computer or a processor. By way of example but not limitation, such non-transitory computer-readable or processor-readable media may include random access memory (RAM), read-only memory (ROM), electrically erasable programmable read-only memory (EEPROM), FLASH memory, disk storage, magnetic storage devices, or the like. Disk storage, as used herein, includes compact disc (CD), laser disc, optical disc, digital versatile disc (DVD), floppy disk, and Blu-ray Disc™, or other storage devices that store data magnetically or optically with lasers. Combinations of the above types of media are also included within the scope of the terms non-transitory computer-readable and processor-readable media. Additionally, any combination of instructions stored on the one or more non-transitory processor-readable or computer-readable media may be referred to herein as a computer program product.

Many modifications and other embodiments of the inventions set forth herein will come to mind to one skilled in the art to which these inventions pertain having the benefit of teachings presented in the foregoing descriptions and the associated drawings. Although the figures only show certain components of the apparatus and systems described herein, it is understood that various other components may be used in conjunction with the supply management system. Therefore, it is to be understood that the inventions are not to be limited to the specific embodiments disclosed and that modifications and other embodiments are intended to be included within the scope of the appended claims. Moreover, the steps in the method described above may not necessarily occur in the order depicted in the accompanying diagrams, and in some cases one or more of the steps depicted may occur substantially simultaneously, or additional steps may be involved. Although specific terms are employed herein, they are used in a generic and descriptive sense only and not for purposes of limitation.

We claim:

1. A system for controlling oxygen supply, the system comprising:
a processor and a memory, wherein the processor is communicably coupled to the memory, a first oxygen supply equipment and a second oxygen supply equipment, wherein the processor is configured to:
determine a pressure of oxygen supply by using one or more sensors coupled to at least one of the first oxygen supply equipment or the second oxygen supply equipment during an inhalation phase; and
compare the pressure of the oxygen supply with a pressure threshold associated with one of the first oxygen supply equipment or the second oxygen supply equipment.
wherein in response to the comparison,
actuate at least a valve coupled to the first oxygen supply equipment or the second oxygen supply equipment to switch the oxygen supply from the first oxygen supply equipment to the second oxygen supply equipment or vice-versa when the oxygen supply falls below the pressure threshold.

2. The system of claim 1, wherein the pressure threshold is indicative of a low-pressure minima associated with first oxygen supply equipment and a high-pressure minimum associated with the second oxygen supply equipment.

3. The system of claim 1, wherein pressure range of the oxygen supply from the first oxygen supply equipment is about approximately 5 to 7 Psi.

4. The system of claim 1, wherein pressure range of the oxygen supply from the first oxygen supply equipment is about approximately 5-30 Psi.

5. The system of claim 1, wherein the processor is further configured to:
determine a pressure drop rate and compare the pressure drop rate with a pressure drop rate threshold; wherein when the pressure drop rate exceeds or is equal to the pressure drop rate threshold; and
switch the oxygen supply from the first oxygen supply equipment to the second oxygen supply equipment or vice-versa.

6. The system of claim 5, wherein the processor is further communicatively coupled to an alarm device and configured to generate an alarm when at least one of:
the pressure falls below the pressure threshold; or
the pressure drop rate exceeds the pressure drop rate threshold.

7. The system of claim 1, wherein the processor is further configured to:
regulate the pressure of the oxygen supply to first oxygen supply equipment and the second oxygen supply equipment via a pressure regulator using feedback from the one or more sensors.

8. The system of claim 1, wherein the memory further stores patient related data and oxygen supply equipment related data, wherein:
the patient related data comprises at least one of patient name, patient age, patient address, patient ailment history and patient treatment history; and
the oxygen supply equipment related data comprises at least one of nominal oxygen content, nominal outlet pressure, flow rate and net weight.

9. The system of claim 8, wherein the processor is further configured to:
select one of the first oxygen supply equipment or the second oxygen supply equipment to be used on the patient based on the patient related data.

10. The system of claim 8, wherein the processor is further configured to:
preset the pressure threshold of the first oxygen supply equipment and the second oxygen supply equipment based on the oxygen supply equipment related data and the patient related data.

11. A method for controlling oxygen supply, the method comprising:
detecting current pressure of a preset oxygen supply during each inhalation phase of a patient, wherein the preset oxygen supply is provided by either a high-flow oxygen supply equipment or a low-flow oxygen supply equipment,
wherein the high-flow oxygen supply equipment is capable of supplying oxygen in a high-pressure range, and
the low-flow oxygen supply equipment is capable of supplying oxygen in a low-pressure range.
comparing the current pressure of the preset oxygen supply with either a low-pressure minimum associated with the low-pressure range, or a high-pressure minimum associated with the high-pressure range;
based on comparison,
switching the preset oxygen supply from the low-flow oxygen supply equipment to the high-flow oxygen supply equipment when the current pressure falls below the low-pressure minima; or
switching the preset oxygen supply from the high-flow oxygen supply equipment to the low-flow oxygen supply equipment when at least one of:
the current pressure falls below the high-pressure minima, and
a pressure drop rate of the current pressure exceeds or is equal to a pressure drop rate threshold.

12. The method of claim 11, wherein both the current pressure falling below the high-pressure minima and the pressure drop rate of the current pressure exceeding the pressure drop rate threshold are indicative of emptying of the high-flow oxygen supply equipment.

13. The method of claim 11, wherein detecting the current pressure further comprises:
detecting the current pressure of the preset oxygen supply by a low-pressure sensor when the preset oxygen supply is provided by the low-flow oxygen supply equipment; and
detecting the current pressure of the preset oxygen supply by a high-pressure sensor when the preset oxygen supply is provided by the high-flow oxygen supply equipment.

14. The method of claim 11, further comprising generating an alarm when at least one of:
the current pressure falls below the low-pressure minima;
the current pressure falls below the high-pressure minima; and
the pressure drop rate exceeds the pressure drop rate threshold.

15. The method as claimed in claim 11, wherein switching between the high-flow oxygen supply equipment and the low-flow oxygen supply equipment further comprises actuating a valve connected with both the high-flow oxygen supply equipment and the low-flow oxygen supply equipment.

16. The method as claimed in claim 11, further comprising selecting the preset oxygen supply based on patient related data, and wherein the patient related data comprises at least one of patient name, patient age, patient address, patient ailment history and patient treatment history.

17. The method as claimed in claim 16, further comprising Selecting one of the high-flow oxygen supply equipment and the low-flow oxygen supply equipment to be used on the patient based on the patient related data.

18. The method as claimed in claim 16, further comprising selecting the preset oxygen supply based on oxygen supply equipment, and wherein the oxygen supply equipment related data comprises at least one of nominal oxygen content, nominal outlet pressure, flow rate and net weight.

19. The method as claimed in claim 11, further comprising regulating the pressure of the oxygen supply to first oxygen supply equipment and the second oxygen supply equipment.

20. The method as claimed in claim 11, further comprising simultaneously generating the alarm and actuating the valve when at least one of:
the current pressure falls below the low-pressure minima;
the current pressure falls below the high-pressure minima; and
the pressure drop rate exceeds or equals the pressure drop rate threshold.
